(19) United States
(12) Patent Application Publication
(10) Pub. No.: US 2023/0240548 A1
(43) Pub. Date: Aug. 3, 2023

(54) Heart Rate Detection Method, Device, and Program

(71) Applicant: Nippon Telegraph and Telephone Corporation, Tokyo (JP)

(72) Inventors: Nobuaki Matsuura, Tokyo (JP); Kei Kuwabara, Tokyo (JP); Yuichi Higuchi, Tokyo (JP); Takako Ishihara, Tokyo (JP)

(21) Appl. No.: 18/007,886

(22) PCT Filed: Jun. 23, 2020
(86) PCT No.: PCT/JP2020/024526

§ 371 Date: Dec. 2, 2022

(51) Int. Cl. A61B 5/0245; A61B 5/339; A61B 5/346; A61B 5/00

(52) U.S. Cl. CPC A61B 5/0245; A61B 5/339; A61B 5/346; A61B 5/7242; A61B 5/7246; A61B 5/7221

(57) ABSTRACT

According to the present disclosure, a heart rate detection method includes measuring an ECG waveform, calculating a heart rate from the ECG waveform, calculating a difference value of a potential of the ECG waveform between sampling time points, calculating an integrated value obtained by integrating the difference value between a measurement time point and any time point before measurement, and determining whether the heart rate at the measurement time point is to be displayed, by comparing the integrated value to a reference value.

### CROSS-REFERENCE TO RELATED APPLICATIONS

This application is a national phase entry of PCT Application No. PCT/JP2020/024526, filed on Jun. 23, 2020, which application is hereby incorporated herein by reference.

### TECHNICAL FIELD

The present disclosure relates to a heart rate detection method, apparatus, and program for presenting an accurate heart rate.

### BACKGROUND

The heart rate or the fluctuation of the heart rate is biological information obtained from an electrocardiogram (ECG), and is utilized for evaluation of autonomic nerve function, an index of exercise intensity, and the like in a daily life and a resting state.

In particular, measuring the heart rate over a relatively long time in a daily life, or the like by using a wearable ECG waveform acquisition/heartbeat measurement device is useful for physical condition management of a user and the like.

PTL 1 discloses a method capable of appropriately detecting a heartbeat in a case where noise is superimposed on an ECG waveform, such as measurement by a wearable device.

Unfortunately, the heartbeat may be erroneously detected in this method in a case where the noise of the ECG waveform due to a body motion is large. For example, in a case where an R wave to be detected is missed, a wave that is not the R wave may be detected as the R wave.

To avoid such an error in heartbeat detection, a wearable heartbeat measurement device may adopt a method of calculating and presenting an average heart rate (referred to as an “HR” below) in which fluctuation of an instantaneous heart rate (referred to as an “IHR” below) caused by an error in heartbeat detection is suppressed by moving-averaging the heart rate over a predetermined time or using an infinite impulse response (IIR) filter, and the like (PTL 2).

### CITATION LIST

### Patent Literature

PTL 1: JP 6527286 B

PTL 2: JP 6645926 B

### SUMMARY

### Technical Problem

In the heartbeat detection method disclosed in PTL 2, a value such as an average heart rate is calculated for any ECG waveform data. Thus, even in a situation where the heartbeat is erroneously detected and thus is not measured as an accurate value, an abnormal numerical value may be presented to the user as an accurate value.

FIG. 10 illustrates a part of an ECG waveform measured by using a wearable device. FIG. 10 also illustrates an R-R interval [ms] corresponding to a heart rate at a timing at which an R wave is detected (black rhombus in FIG. 10). Here, for example, the method disclosed in PTL 1 can be used to detect the R-R interval.

In the method disclosed in PTL 1, variation in the value of the R-R interval is also observed together with the disturbance of the ECG waveform, and thus it is not possible to present an accurate heart rate.

Next, an instantaneous heart rate IHR and an average heart rate HR calculated based on the R-R interval in the method disclosed in PTL 2 will be described.

FIG. 11 illustrates an instantaneous heart rate IHR and an average heart rate HR calculated based on the instantaneous heart rate IHR. In FIG. 11, each black rhombus indicates the instantaneous heart rate, and each white circle indicates the average heart rate.

The instantaneous heart rate IHR is calculated by the equation: the instantaneous heart rate IHR [bpm]=60,000÷(R-R interval [ms]).

In addition, the average heart rate HR [n] is calculated by the following equation with respect to time-series data IHR [n] of the instantaneous heart rate by using the IIR filter.

HR[n]=(1−a)×HR[n−1]+a×IHR[n]

Here, a is equal to 0.1.

As illustrated in FIG. 11, in this method, the average heart rate having a substantially fixed value is observed.

FIG. 12 illustrates measurement results of triaxial accelerations simultaneously measured by an accelerometer incorporated in the wearable device used to measure the ECG waveform described above. In FIG. 12, a solid line 51 indicates an x-axis acceleration, a broken line 52 indicates a y-axis acceleration, and a dotted line 53 indicates a z-axis acceleration. It is understood that the device is largely vibrated in the y axis and the z axis thereof in the above-described measurement.

This indicates that the disturbance of the ECG waveform illustrated in FIG. 10 is caused by a change in contact resistance between an electrode and the skin due to the influence of this acceleration. At this time, the R wave is not accurately recognized, and a change in the waveform influenced by the acceleration is erroneously detected as the R wave.

As described above, in an environment in which the ECG waveform illustrated in FIG. 10 is measured, the R wave is not accurately detected due to the vibration. Thus, the instantaneous heart rate and the average heart rate calculated based on the R-R interval in FIG. 11 do not have accurate values. Unfortunately, in FIG. 11, the average heart rate has a stable measurement value.

In this case, since the average heart rate has the stable measurement value, a user may recognize the average heart rate, which is not accurate, as an accurate value.

As described above, in the heartbeat detection method in the related art, even in a situation where the heartbeat is erroneously detected and is not measured as an accurate value, an abnormal numerical value may be presented to the user as the accurate value. Thus, to present an accurate heart rate, an index instead of the average heart rate is required.

### Means for Solving the Problem

To solve the problem as described above, according to the present disclosure, there is provided a heart rate detection method including measuring an ECG waveform, calculating a heart rate from the ECG waveform, calculating a difference value of a potential of the ECG waveform between sampling time points, calculating an integrated value obtained by integrating the difference value between a measurement time point and any time point before measurement, and determining whether the heart rate at the measurement time point is to be displayed, by comparing the integrated value to a reference value.

In addition, according to the present disclosure, a heart rate detection apparatus includes a measurement unit configured to measure an ECG waveform, a calculation unit configured to calculate a heart rate from the ECG waveform, calculate a difference value of a potential of the ECG waveform between sampling time points, and calculate an integrated value obtained by integrating the difference value between a measurement time point and any time point before measurement, and a determination unit configured to determine whether the heart rate at the measurement time point is to be displayed, by comparing the integrated value to a reference value.

In addition, according to the present disclosure, there is provided a heart rate detection program causing a heart rate detection apparatus to operate, the heart rate detection program causing the heart rate detection apparatus to perform measuring an ECG waveform, calculating a heart rate from the ECG waveform, calculating a difference value of a potential of the ECG waveform between sampling time points, calculating an integrated value obtained by integrating the difference value between a measurement time point and any time point before measurement, and determining whether the heart rate at the measurement time point is to be displayed, by comparing the integrated value to a reference value.

### Effects of Embodiments of the Invention

According to the present disclosure, it is possible to provide a heart rate detection method, apparatus, and program for presenting an accurate heart rate.

### BRIEF DESCRIPTION OF THE DRAWINGS

FIG. 1 is a diagram illustrating an ECG waveform for describing a heart rate detection method according to a first embodiment of the present disclosure.

FIG. 2 is a diagram illustrating a temporal change of an index in the heart rate detection method according to the first embodiment of the present disclosure.

FIG. 3 is a diagram illustrating the ECG waveform and an R-R interval for describing the heart rate detection method according to the first embodiment of the present disclosure.

FIG. 4 is a diagram illustrating the temporal change of the index in the heart rate detection method according to the first embodiment of the present disclosure.

FIG. 5 is a diagram illustrating a temporal change of an index in a method in the related art.

FIG. 6 is a block diagram illustrating a heart rate detection apparatus according to the first embodiment of the present disclosure.

FIG. 7 is a flowchart illustrating the heart rate detection method according to the first embodiment of the present disclosure.

FIG. 8 is a flowchart illustrating a heart rate detection method according to a second embodiment of the present disclosure.

FIG. 9 is a diagram illustrating a configuration example of a computer in the embodiments of the present disclosure.

FIG. 10 is a diagram illustrating an ECG waveform and an R-R interval for describing a heart rate detection method in the related art.

FIG. 11 is a diagram illustrating an instantaneous heart rate and an average heart rate for describing the heart rate detection method in the related art.

FIG. 12 is a diagram illustrating triaxial accelerations in a wearable device for describing the heart rate detection method in the related art.

### DETAILED DESCRIPTION OF ILLUSTRATIVE EMBODIMENTS

As described above, to present an accurate heart rate, an index instead of the average heart rate is required. In the present disclosure, a numerical value indicating the disturbance of an ECG waveform is used as a new index.

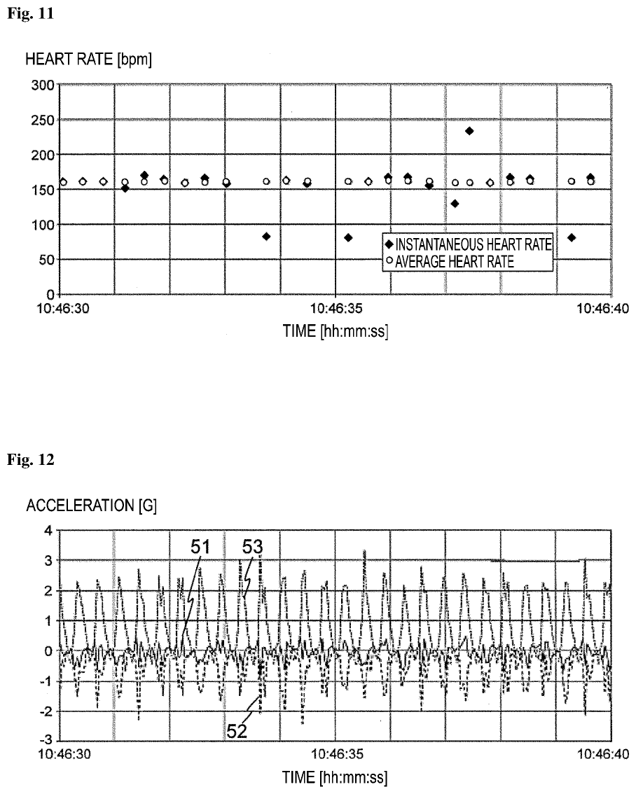

As described above, in the ECG waveform in FIG. 10, the potential greatly fluctuates beyond a normal amplitude (about 3 mV) of an ECG waveform. As illustrated in FIG. 11, an instantaneous heart rate extracted from this waveform has low reliability even when the variation is small. Thus, in the present disclosure, the disturbance itself of the ECG waveform is quantified and used as an index.

In the present disclosure, as the index, a value obtained by integrating the change amount (referred to as a “time difference value” below) of the potential of an ECG waveform (referred to as an “ECG potential” below) for each sampling period in a predetermined period is used. Here, the change amount (time difference value) for each sampling period is set to |x[n]−x[n−1]|, when the ECG potential at a certain time point t[n] is set as x[n].

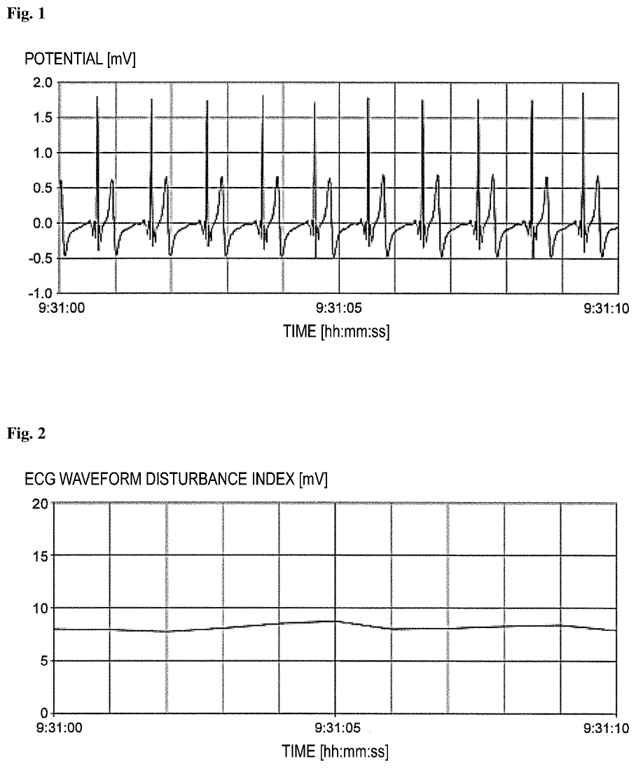

First, the index described above is calculated for a standard ECG waveform having no noise, which is illustrated in FIG. 1. The sampling period (interval) in measurement of the ECG waveform is 5 ms.

For the standard ECG waveform, the time difference value is integrated, and the obtained integrated value is used as the index. The integration time is set to one second in the past.

FIG. 2 illustrates a temporal change of the integrated value being the index described above. The integrated value being the index is substantially fixed at approximately 10 mV or less.

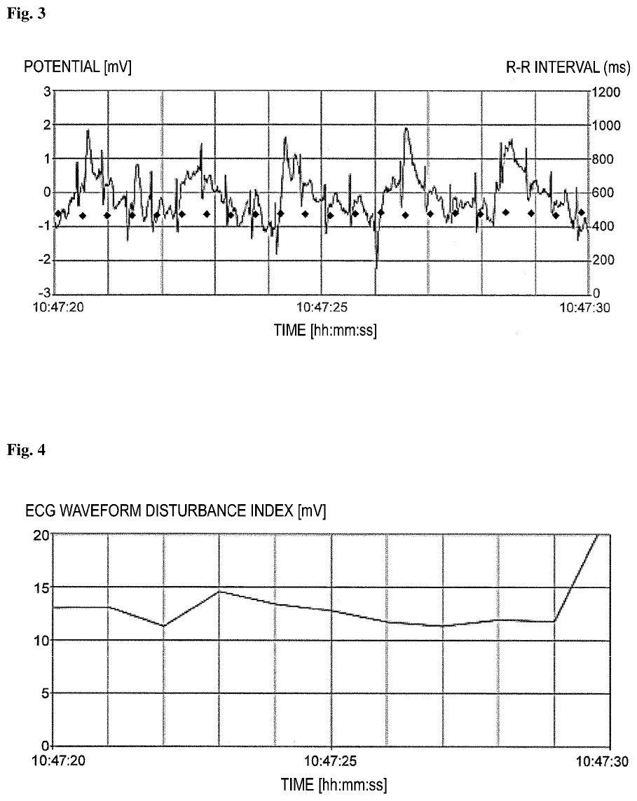

Then, an ECG waveform having suppressed noise is assumed as an example of an ECG waveform to be practically measured, and the index described above is calculated. FIG. 3 illustrates an ECG waveform including fluctuation of a baseline, as an example of the ECG waveform having the suppressed noise. The sampling period (interval) is 5 ms. FIG. 3 also illustrates an R-R interval [ms] at a timing at which the R wave is detected.

Although the disturbance of the ECG waveform is observed, the R wave is stably observed at a substantially fixed value, and is detected as an accurate value.

The time difference value for this ECG waveform is integrated, and the obtained integrated value is used as the index. The integration time is set to one second in the past.

FIG. 4 illustrates a temporal change of the integrated value being the index. The integrated value being the index is substantially fixed at a voltage equal to or lower than at least approximately 20 mV.

From the above description, it is understood that a reference value for determining whether an R wave can be detected by the disturbance of the ECG waveform is appropriate at 20 mV.

The present disclosure is based on the knowledge that an accurate heart rate can be presented when an index obtained by integrating a change amount (time difference value) of an ECG potential for each sampling period is equal to or lower than the reference value of 20 mV.

Here, a method of using a change amount of a potential of an ECG waveform for each sampling period for determining whether the size of a wearable device is appropriate is disclosed (JP 2018-183412 A). In this method, the reference value used in the determination is 500 mV (sampling interval: 5 ms) with respect to the integrated value of the change amount for 5 seconds, that is, 100 mV per second.

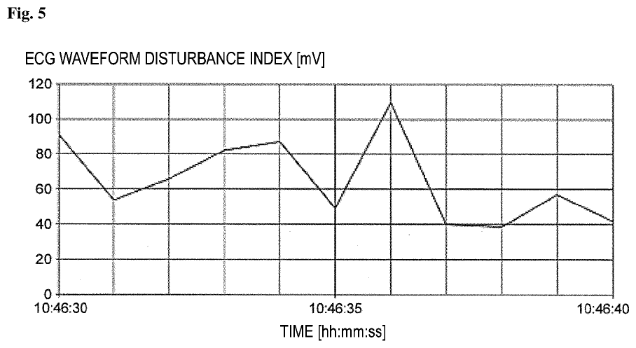

In a case of determining the size of the wearable device, a case where an electrode is detached is expected. Thus, a reference value is set assuming large waveform disturbance in which the ECG waveform goes off scale. FIG. 5 illustrates an example of an index used to determine the size of a wearable device.

In FIG. 5, for the ECG waveform illustrated in FIG. 10, an integrated value of the change amount of a potential for one second in the past is plotted as the index. The index is equal to or lower than 100 mV being the reference value in most of the measurement time. Thus, it is determined that the size of the wearable device is accurate.

As described above, such a method is different from the heart rate detection method according to the embodiment of the present disclosure in terms of a target to be inspected (determined), and thus the reference value used for the inspection (determination) is also greatly different.

### First Embodiment

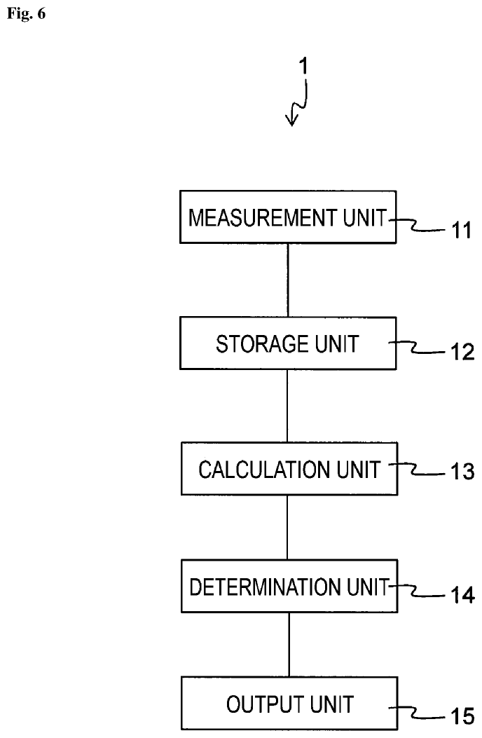

A heart rate detection apparatus and method according to a first embodiment of the present disclosure will be described with reference to FIGS. 6 to 8.

### Configuration of Heart Rate Detection Apparatus

FIG. 6 is a block diagram illustrating an example of a configuration of a heart rate detection apparatus 1 according to the present embodiment. The heart rate detection apparatus 1 includes a measurement unit 11, a storage unit 12, a calculation unit 13, a determination unit 14, and an output unit 15.

The measurement unit 11 measures an ECG waveform, and uses an electrocardiograph in a wearable device as an example.

The storage unit 12 stores a time point and an ECG potential measured by the electrocardiograph.

The calculation unit 13 acquires the time point and the ECG potential measured by the electrocardiograph from the storage unit 12, calculates a time difference value, and calculates an integrated value thereof. The calculation unit 13 performs calculation based on the ECG waveform in order to detect a heart rate (R-R interval).

The determination unit 14 determines whether (validity) the heart rate acquired for the time for integration is accurate, by comparing the calculated integrated value of the time difference value of the ECG potential to a reference value.

The output unit 15 outputs the calculated heart rate. Information indicating that the heart rate has not been accurately measured, the measured ECG waveform, the time difference value, and the integrated value can also be output.

### Heart Rate Detection Method

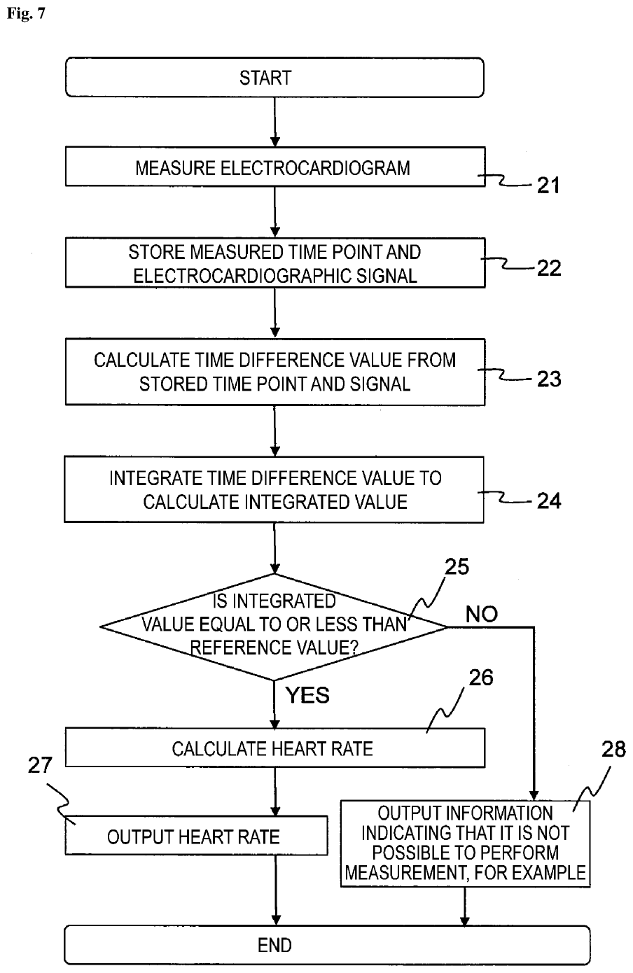

FIG. 7 is a flowchart illustrating the heart rate detection method according to the present embodiment.

First, an ECG waveform is measured (Step 21).

Then, the measured time point and the measured ECG waveform (ECG potential) are stored (Step 22).

Then, a time difference value is calculated by using the stored ECG waveform (ECG potential) (Step 23). The time difference value refers to a difference |x[n]−x[n−1]| between the ECG potential x[n] measured by the measurement unit 11 at a certain time point t[n] and the ECG potential x[n−1] measured at the latest time point t[n−1] before the measurement and acquired from the storage unit 12. In other words, the time difference value is a difference value of the ECG waveform (ECG potential) between sampling time points.

Then, an integrated value is calculated by integrating the time difference value between the measurement time point t[n] and the certain time point t[n−m] before the measurement (referred to as an “integration time” below) (Step 24).

Then, the integrated value is compared to the reference value, and determination is performed as follows (Step 25).

When the integrated value is equal to or less than the reference value, it is determined that the heart rate calculated from the ECG waveform in the integration time including the measurement time point t[n] is accurate.

In this case, the heart rate at the measurement time point t[n] is calculated from the ECG waveform (Step 26). Here, the heart rate (R-R interval) can be calculated, for example, by being compared to a predetermined threshold value based on a change in a time difference value of the ECG waveform. It is possible to improve measurement accuracy, for example, by comparing the heart rate to a predetermined threshold value by using changes in time difference values of a plurality of ECG waveforms (see PTL 1).

Finally, the calculated heart rate is output (presented) (Step 27).

When the integrated value is more than the reference value, it is determined that the heart rate calculated from the ECG waveform in the integration time is not accurate.

In this case, the heart rate is not calculated, and information indicating that an accurate heart rate is not measured is output (presented) (Step 28). Alternatively, the latest accurate heart rate before the measurement time point t[n] may be presented, or a blank (state where nothing is displayed) may be presented.

Next, an example of an operation (output) in the heart rate detection method and apparatus according to the present embodiment will be described. Here, the reference value is 20 mV.

In a case where the ECG waveform illustrated in FIG. 1 is measured, the index illustrated in FIG. 2 is calculated. Since the calculated index is equal to or less than 20 mV, it is determined that the heart rate calculated from the ECG waveform is accurate. Thus, the heart rate calculated from the ECG waveform illustrated in FIG. 1 is output (presented).

Similarly, in a case where the ECG waveform illustrated in FIG. 3 is measured, the index illustrated in FIG. 4 is calculated. Since the calculated index is equal to or less than 20 mV, it is determined that the heart rate calculated from the ECG waveform is accurate. Thus, the heart rate calculated from the ECG waveform illustrated in FIG. 3 is output (presented).

In a case where the ECG waveform illustrated in FIG. 10 is measured, the index illustrated in FIG. 5 is calculated. Since the calculated index is equal to or more than 20 mV, it is determined that the heart rate calculated from the ECG waveform is not accurate. Thus, information indicating that an accurate heart rate is not measured is output (presented). Alternatively, the latest accurate heart rate before the measurement time point is presented. Alternatively, a blank (state in which nothing is displayed) is presented.

As described above, according to the present embodiment, it is possible to avoid presenting an abnormal numerical value to the user in a situation where the heart rate is not accurately measured, and to present only an accurate numerical value of the heart rate to the user.

### Second Embodiment

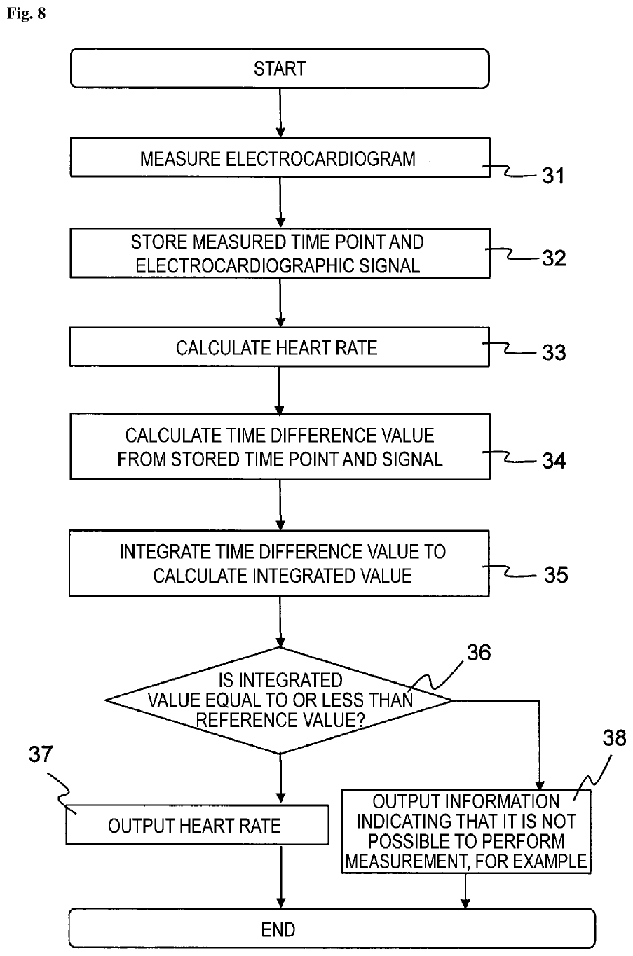

A heart rate detection apparatus and method according to a second embodiment of the present disclosure will be described with reference to FIG. 8. A configuration of the heart rate detection apparatus according to the second embodiment is similar to the configuration of the first embodiment. In the first embodiment, an example has been described in which it is determined that the measured heart rate is accurate, and then the heart rate is calculated. In the second embodiment, the heart rate is calculated before the determination.

### Heart Rate Detection Method

FIG. 8 is a flowchart illustrating the heart rate detection method according to the present embodiment.

First, an ECG waveform is measured (Step 31).

Then, the measured time point and the measured ECG waveform (ECG potential) are stored (Step 32).

Then, the heart rate is calculated from the ECG waveform and stored (Step 33). Here, the heart rate (R-R interval) can be calculated, for example, by being compared to a predetermined threshold value based on a change in a time difference value of the ECG waveform. It is possible to improve measurement accuracy, for example, by comparing the heart rate to a predetermined threshold value by using changes in time difference values of a plurality of ECG waveforms (see PTL 1).

Then, a time difference value is calculated by using the stored ECG waveform (ECG potential) (Step 34). The time difference value refers to a difference |x[n]−x[n−1]| between the ECG potential x[n] measured by the measurement unit 11 at a certain time point t[n] and the ECG potential x[n−1] measured at the latest time point t[n−1] before the measurement and acquired from the storage unit 12.

Then, an integrated value is calculated by integrating the time difference value between the measurement time point t[n] and a certain time point t[n−m] before the measurement (referred to as an “integration time” below) (Step 35).

Then, the integrated value is compared to the reference value, and determination is performed as follows (Step 36).

When the integrated value is equal to or less than the reference value, it is determined that the heart rate calculated from the ECG waveform in the integration time is accurate.

In this case, the heart rate calculated from the ECG waveform measured in the integration time is acquired from the storage unit 12 and output (presented) (Step 37).

When the integrated value is more than the reference value, it is determined that the heart rate calculated from the ECG waveform in the integration time is not accurate.

In this case, information indicating that an accurate heart rate is not measured is output (displayed) (Step 38). Alternatively, the latest accurate heart rate may be displayed, or nothing may be displayed.

The heart rate detection method according to the present embodiment can exhibit effects similar to the effects of the heart rate detection method according to the first embodiment. Thus, it is possible to avoid presenting an abnormal numerical value to the user in a situation where the heart rate is not accurately measured, and to present only an accurate numerical value of the heart rate to the user.

The heart rate detection apparatus according to the present embodiment may be worn on the body of the user as a wearable device.

Alternatively, in the heart rate detection apparatus according to the present embodiment, the measurement unit may be worn on the body of the user as a wearable device, and the storage unit, the calculation unit, and the determination unit may be provided in a smartphone, a server, or the like outside the wearable device. In this case, the heart rate detection apparatus includes a transmission/reception unit in each of the wearable device, an external server, and the like, transmits an ECG waveform measured by the wearable device to the server and the like, and causes the server and the like to perform storing, calculation, determination, and the like. Finally, the heart rate and the like (including information indicating that the heart rate is not measured) may be output to the server and the like, or may be transmitted to the wearable device or the like and output.

In the embodiments according to the present disclosure, the examples have been described in which the latest ECG waveform (ECG potential) is sequentially acquired from the storage unit at a timing of measurement, and the validity of whether the heart rate is accurate is determined. The ECG waveforms may be collectively stored, and then acquired from the storage unit, and the validity of the heart rate may be determined.

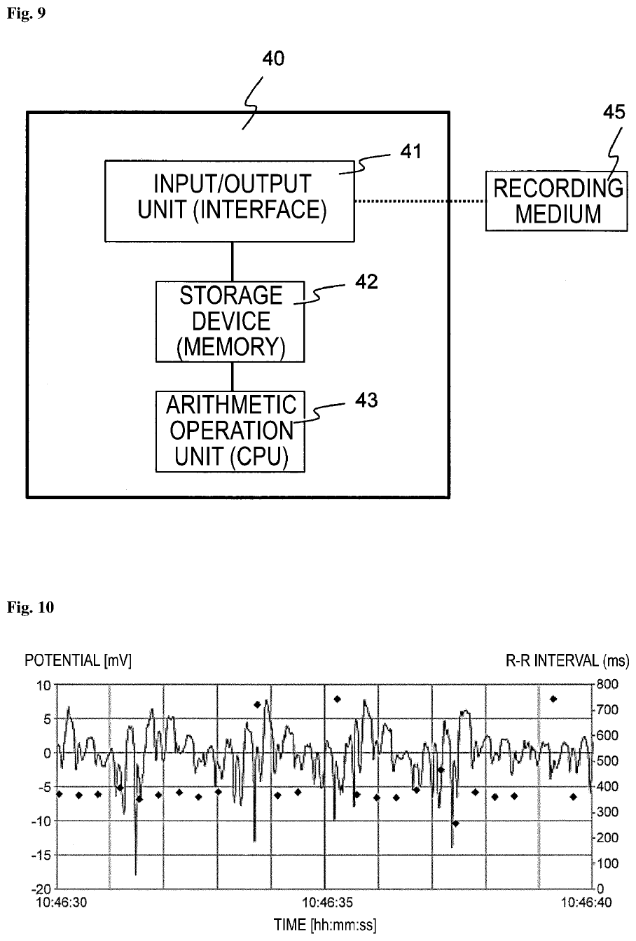

FIG. 9 illustrates a configuration example of a computer 40 in the heart rate detection apparatus according to the embodiments of the present disclosure. The heart rate detection apparatus can be achieved by the computer 40 including a central processing unit (CPU) 43, a storage device (storage unit) 42, and an interface device 41, and programs for controlling the hardware resources. Here, the measurement unit and the output unit in the heart rate detection apparatus according to the embodiments of the present disclosure are connected to the interface device 41. The CPU 43 executes processing in the embodiments of the present disclosure, in accordance with a heart rate detection program stored in the storage device 42. In this manner, the heart rate detection program causes the heart rate detection apparatus to operate.

In the heart rate detection apparatus according to the embodiments of the present disclosure, the computer may be provided inside the device, or at least one of the functions of the computer may be implemented by using an external computer. A storage medium outside the apparatus may also be used as the storage unit, and a heart rate detection program stored in the storage medium may be read and executed. The storage medium includes various magnetic recording media, magneto-optical recording media, CD-ROMs, CD-Rs, and various memories. The heart rate detection program may be supplied to the computer via a communication line such as the Internet.

In the embodiments of the present disclosure, an example of the structure, dimension, material, and the like of each component in the configuration of the heart rate detection apparatus, the heart rate detection method, and the like has been described; however, the present disclosure is not limited thereto. Any structure, dimension, material, and the like may be available as long as the apparatus exhibits the functions and effects of the heart rate detection apparatus and method.

### INDUSTRIAL APPLICABILITY

The present disclosure can be applied to a technique for analyzing a biological signal obtained from an ECG waveform.

### REFERENCE SIGNS LIST

1 Heart rate detection apparatus

11 Measurement unit

12 Storage unit

13 Calculation unit

14 Determination unit

15 Output unit.

1-7. (canceled)

8. A heart rate detection method comprising:
measuring, by a heart rate detection apparatus, an ECG waveform at a measurement time point;
calculating a heart rate from the ECG waveform;
calculating a difference value of a potential of the ECG waveform between sampling time points;
calculating an integrated value obtained by integrating the difference value between the measurement time point and a time point before the measurement time point; and
determining whether the heart rate at the measurement time point is to be displayed, by comparing the integrated value to a reference value.

9. The heart rate detection method according to claim 8, wherein the reference value is 20 mV.

10. The heart rate detection method according to claim 8, wherein determining whether the heart rate at the measurement time point is to be displayed comprises:
when the integrated value is equal to or less than the reference value, determining to display the heart rate at the measurement time point; and
when the integrated value is more than the reference value, determining to display a blank=.

11. The heart rate detection method according to claim 8, wherein determining whether the heart rate at the measurement time point is to be displayed comprises:
when the integrated value is equal to or less than the reference value, determining to display the heart rate at the measurement time point; and
when the integrated value is more than the reference value, determining to display information indicating that an accurate heart rate is not measured.

12. The heart rate detection method according to claim 8, wherein determining whether the heart rate at the measurement time point is to be displayed comprises:
when the integrated value is equal to or less than the reference value, determining to display the heart rate at the measurement time point; and
when the integrated value is more than the reference value, determining to display a latest accurate heart rate that is determined before the measurement time point.

13. A heart rate detection apparatus comprising:
a measurement circuit configured to measure an ECG waveform at a measurement time point;
a calculation circuit configured to:
calculate a heart rate from the ECG waveform;
calculate a difference value of a potential of the ECG waveform between sampling time points; and
calculate an integrated value obtained by integrating the difference value between the measurement time point and a time point before the measurement time point; and
a determination circuit configured to determine whether the heart rate at the measurement time point is to be displayed, by comparing the integrated value to a reference value.

14. The heart rate detection apparatus according to claim 13, wherein the reference value is 20 mV.

15. The heart rate detection apparatus according to claim 13, wherein:
when the integrated value is equal to or less than the reference value, the determination circuit is configured to determine to display the heart rate at the measurement time point; and
when the integrated value is more than the reference value, the determination circuit is configured to determine to display a blank.

16. The heart rate detection apparatus according to claim 13, wherein:
when the integrated value is equal to or less than the reference value, the determination circuit is configured to determine to display the heart rate at the measurement time point; and
when the integrated value is more than the reference value, the determination circuit is configured to determine to display information indicating that an accurate heart rate is not measured.

17. The heart rate detection apparatus according to claim 13, wherein:
when the integrated value is equal to or less than the reference value, the determination circuit is configured to determine to display the heart rate at the measurement time point; and
when e the integrated value is more than the reference value, the determination circuit is configured to determine to display a latest accurate heart rate that is determined before the measurement time point.

18. A non-transitory memory storing a heart rate detection program causing a heart rate detection apparatus to operate, the heart rate detection program causing the heart rate detection apparatus to perform:
measuring an ECG waveform at a measurement time point;
calculating a heart rate from the ECG waveform;
calculating a difference value of a potential of the ECG waveform between sampling time points;
calculating an integrated value obtained by integrating the difference value between the measurement time point and a time point before the measurement time point; and
determining whether the heart rate at the measurement time point is to be displayed, by comparing the integrated value to a reference value.

19. The non-transitory memory storing the heart rate detection program according to claim 18, wherein the reference value is 20 mV.

20. The non-transitory memory storing the heart rate detection program according to claim 18, wherein the heart rate detection program further causes the heart rate detection apparatus to perform:
when the integrated value is equal to or less than the reference value, determine to display the heart rate at the measurement time point; and
when the integrated value is more than the reference value, determine to display a blank.

21. The non-transitory memory storing the heart rate detection program according to claim 18, wherein the heart rate detection program further causes the heart rate detection apparatus to perform:
when the integrated value is equal to or less than the reference value, determine to display the heart rate at the measurement time point; and
when the integrated value is more than the reference value, determine to display information indicating that an accurate heart rate is not measured.

22. The non-transitory memory storing the heart rate detection program according to claim 18, wherein the heart rate detection program further causes the heart rate detection apparatus to perform:
when the integrated value is equal to or less than the reference value, determine to display the heart rate at the measurement time point; and
when e the integrated value is more than the reference value, determine to display a latest accurate heart rate that is determined before the measurement time point.
